(19) United States
(12) Patent Application Publication
(10) Pub. No.: US 2021/0169497 A1
(43) Pub. Date: Jun. 10, 2021

(54) MEDICAL MATERIAL

(71) Applicant: GUNZE LIMITED, Ayabe-shi, Kyoto (JP)

(72) Inventors: Yuki SAKAMOTO, Ayabe-shi (JP); Hideki SATO, Ayabe-shi (JP); Saki OKUMURA, Ayabe-shi (JP)

(73) Assignee: GUNZE LIMITED, Ayabe-shi, Kyoto (JP)

(21) Appl. No.: 17/257,623

(22) PCT Filed: Mar. 26, 2019
(86) PCT No.: PCT/JP2019/012747

§ 371 Date: Jan. 4, 2021

(30) Foreign Application Priority Data
Jul. 12, 2018 (JP) 2018-132538

(51) Int. Cl. A61B 17/12; A61B 17/00

(52) U.S. Cl. CPC A61B 17/12122; A61B 17/0057; A61B 17/1215; A61B 2017/00623; A61B 2017/00004; A61B 2017/00606; A61B 2017/00592

(57) ABSTRACT

A defect hole closing material achieves low invasive treatment for an atrial septal defect with almost no fear of long-term failure. The defect hole closing material is formed by two cylindrical bodies (a first cylinder part and a second cylinder part) made of a bioabsorbable material and having stitch-like structures, has a sandglass shape, and includes a coil spring both ends of which are respectively engaged with a first end part and a second end part and that is inserted through inner parts of the first cylinder part and the second cylinder part from the first end part side to the second end part side via a substantially central part. A first porous layer is provided on the substantially central part side in the first cylinder part, a second porous layer is provided on the opposite side to the substantially central part in the second cylinder part, and shape holding members that hold the shape against force acting along the lengthwise direction are provided on both end sides of the defect hole closing material.

### TECHNICAL FIELD

The present invention relates to a medical material for treating a defect hole formed in a biological tissue, and in particular, to a medical material that is set in a catheter, sent to a treatment site through a blood vessel, and placed in a living body.

### BACKGROUND ART

The heart of a human is divided into left and right chambers by a tissue called septum, in which each of the left and the right chambers has an atrium and a ventricle. That is, the heart is configured by two atria and two ventricles, i.e., a right atrium, a right ventricle, a left atrium, and a left ventricle. As for the heart having such a configuration, there is known an atrial septal defect (ASD) that is a disorder caused by developmental difficulty in a fetal stage, wherein a hole called a defect hole congenitally opens in an atrial septum partitioning the right atrium and the left atrium.

As treatment for the atrial septal defect, the following two methods exist. One is a surgical operation that is performed with chest cut, and the other one is catheterization using an occluder without cutting the chest.

The surgical operation (patch operation) uses a cardiopulmonary bypass, opens the chest, and closes the defect hole by a patch. The catheterization sets the occluder in a catheter, inserts the catheter into a blood vessel, sends the catheter to a target position (defect hole), and then, releases the occluder to place it in the body. The catheterization is performed in such a manner that without incising the chest, a small jig (device) called the occluder, which is folded into an elongated shape, is sent from a vein (femoral vein) at the root of a leg to a position of the hole opening in the atrial septum to occlude the hole. The advantage of the catheterization is that the treatment can be performed by making a tiny skin incision (only a few millimeters) at the base of the leg (inguinal region) which is an inconspicuous body area without performing a thoracotomy requiring general anesthesia.

Japanese Unexamined Patent Application Publication No. 2008-512139 (Patent Literature 1) discloses an assembly (occluder) used for catheterization for the atrial septal defect. This assembly seals a passageway (defect hole) in the heart. The assembly includes: a closure device for hermetically sealing the passageway of the heart that includes a first anchor adapted to be placed proximate a first end of the passageway, a second anchor adapted to be placed proximate a second end of the passageway, and a flexible extension material adapted to extend through the passageway and be connected to the first and second anchors, the second anchor being movable relative to the flexible extension material to change a length of the flexible extension material between the first and second anchors; and a supply system for delivering the closure device to the passageway of the heart, a supply device being designed to move within a lumen of a guide catheter and including a wire for controlling movement of the second anchor along the flexible extension material.

Patent Literature 1 discloses that a patent foramen oval (PFO) closure device (occluder) includes a left atrial anchor, a right atrial anchor, a tether, and a lock, and the left atrial anchor, the right atrial anchor coupled to the left atrial anchor via the tether, and the lock remain in the heart to seal the PFO.

### CITATION LIST

### Patent Literature

Patent Literature 1: Japanese Unexamined Patent Application Publication No. 2008-512139

### SUMMARY OF INVENTION

### Technical Problem

The patch operation involves usage of the cardiopulmonary bypass and poses high invasiveness, resulting in the problem of a long hospitalization period. In contrast, the catheterization is preferable because it does not use the cardiopulmonary bypass and poses low invasiveness, which is conducive to reduction in the hospitalization period.

As disclosed in Patent literature 1, the left atrium anchor and the right atrium anchor remain in the heart. Each of the left atrium anchor and the right atrium anchor includes equal to or more than one arm, and the arm(s) extend(s) radially outwardly from a hub. The arm is preferably formed by a rolled sheet made of a nickel-titanium two-component alloy. A defect hole is occluded by expanding the left atrium anchor and the right atrium anchor in a living body. In this case, the expansion of the anchors is once started, it is difficult to cause the anchors to recover their original states. As disclosed in Patent literature 1, the anchors have to be folded by means of a dedicated retrievable device which has a complicated structure and is difficult to operate from outside the living body.

However, for example, in the event that the anchor is accidentally caught in a biological tissue within the atrium to damage the biological tissue, there may be no time enough to fold the anchor by such a dedicated retrievable device. In this case, there is no other choice but to start the thoracotomy immediately. This finally results in the problem of undergoing the highly invasive thoracotomy.

Further, the defect hole occluder made of metal remains in the body through the whole life, and there is therefore the problem of fear of long-term failure.

The present invention has been made in view of the above-mentioned problems in the conventional art, and an object thereof is to provide a medical material that is capable of being released and placed at a treatment site in a living body, enables low invasive catheterization with easy and accurate operations without a complicated structure, and has almost no fear of long-term failure even when remaining in the body.

### Solution to Problem

In order to accomplish the above object, the medical material according to an aspect of the present invention takes the following technical means.

That is, according to an aspect of the present invention, there is provided a medical material that is formed by a cylindrical body having a stitch-like structure using a filamentary material, wherein the medical material has such a shape that a cylinder diameter of a substantially central part of the cylindrical body is smaller than cylinder diameters of the other parts, a first cylinder part on a first end part side as one end of the cylindrical body in a lengthwise direction and a second cylinder part on a second end part side as the other end of the cylindrical body are formed with the substantially central part as a center, an elastic member both ends of which are respectively engaged with the filamentary material at the first end part and the filamentary material at the second end part and that is inserted through inner parts of the first cylinder part and the second cylinder part from the first end part side to the second end part side via the substantially central part is included, at least any one of a first porous layer arranged on the substantially central part side in the first cylinder part and a second porous layer arranged on an opposite side to the substantially central part in the second cylinder part is arranged on an inner surface of the cylindrical body, the first porous layer and the second porous layer being formed by any one of a nonwoven fabric, sponge, film, and composite body of them, and a shape holding member that is provided at at least one of both ends of the elastic member, is coupled to an end part of the elastic member, and holds the shape of the medical material against force acting along the lengthwise direction for moving the medical material is included.

It is preferable that the shape holding member can include an exterior part formed by winding a yarn into a spiral shape.

It is further preferable that the shape holding member can include an exterior part formed by winding a yarn into a spiral shape and an insertion yarn inserted so as to pass through a center of the spiral shape.

It is still further preferable that when the shape holding members are provided at both ends of the elastic member, thicknesses of the yarns forming the exterior parts can be made different.

It is still further preferable that when the medical material is contained in a catheter, the yarn forming the exterior part in the shape holding member on a root side can be made thicker than the yarn forming the exterior part in the shape holding member on a tip side.

It is still further preferable that in at least any one of the cylinder parts, the insertion yarn can couple the filamentary material and the porous layer.

It is still further preferable that the end part of the elastic member can be joined to a small cylinder part provided outside the cylindrical body having the stitch-like structure and capable of being engaged with an operation wire, and the shape holding member can be coupled to the small cylinder part.

It is still further preferable that the shape can be a sandglass shape, figure-of-eight shape, or double spindle shape, and the porous layers can have umbrella shapes along the shape.

It is still further preferable that when the elastic member is in a compressed state, the first end part and the second end part can come close to each other with the substantially central part as a center, the cylinder diameters of the other parts can be increased to sizes corresponding to a defect hole to be closed by the medical material, and the porous layers can expand to sizes corresponding to the defect hole to be closed by the medical material with increase in the cylinder diameters of the other parts.

It is still further preferable that when the elastic member is in a stretched state, the first end part and the second end part can be separated from each other with the substantially central part as the center, the cylinder diameters of the other parts can be decreased to sizes corresponding to a catheter in which the medical material is contained, and the porous layers can contract to sizes corresponding to the catheter in which the medical material is contained with decrease in the cylinder diameters of the other parts.

It is still further preferable that the elastic member can be formed by a coil spring having a smaller diameter than the cylinder diameter of the substantially central part.

It is still further preferable that the filamentary material or the porous layers can be made of a bioabsorbable material.

### Advantageous Effects of Invention

The medical material according to the present invention is capable of being released and placed at a treatment site in a living body and enables low invasive catheterization with easy and accurate operations without a complicated structure. Furthermore, the medical material according to the present invention has almost no possibility of long-term failure even when remaining in the body.

### BRIEF DESCRIPTION OF DRAWINGS

FIG. 1 is an overall view of a defect hole closing material 100 as an example of a medical material according to the present invention (when a coil spring is in a compressed state).

FIG. 2 is an overall view of the defect hole closing material 100 (when the coil spring is in an intermediate state).

FIG. 3 is an overall view of the defect hole closing material 100 (when the coil spring is in a stretched state).

FIG. 4 is an overall view of the defect hole closing material 100 (when the coil spring is in the compressed state and in the stretched state).

FIG. 5A is a partial side view of the defect hole closing material 100 in FIG. 2.

FIG. 5B is a cross-sectional view along A-A in FIG. 5A.

FIG. 6 is a conceptual view when the defect hole closing material 100 is used for catheterization for an atrial septal defect.

FIG. 7 is an enlarged view (part 1) of a part B in FIG. 6 illustrating the procedure of the catheterization.

FIG. 8 is an enlarged view (part 2) of the part B in FIG. 6 illustrating the procedure of the catheterization.

FIG. 9 is an enlarged view (part 3) of the part B in FIG. 6 illustrating the procedure of the catheterization.

FIG. 10 is an enlarged view of a root-side end part of the defect hole closing material 100.

FIG. 11 is an enlarged view of the coil spring configuring the defect hole closing material 100 in the compressed state.

FIG. 12 is an enlarged view of the coil spring configuring the defect hole closing material 100 in the intermediate state or the stretched state.

FIG. 13A is a view (part 1) for explaining a shape holding member configuring the defect hole closing material 100.

FIG. 13B is a view (part 2) for explaining the shape holding member configuring the defect hole closing material 100.

FIG. 14A is an enlarged view of a tip-side end part of the defect hole closing material 100.

FIG. 14B is a perspective view of FIG. 14A.

FIG. 15A is a view (part 1) for explaining a coupled state between the shape holding members and cylindrical metal pieces at coil spring end parts, which configure the defect hole closing material 100.

FIG. 15B is a view (part 2) for explaining the coupled state between the shape holding members and the cylindrical metal pieces at the coil spring end parts, which configure the defect hole closing material 100.

FIG. 15C is a view (part 3) for explaining the coupled state between the shape holding members and the cylindrical metal pieces at the coil spring end parts, which configure the defect hole closing material 100.

FIG. 16A is a view (part 1) for explaining an action effect of the shape holding members configuring the defect hole closing material 100.

FIG. 16B is a view (part 2) for explaining the action effect of the shape holding members configuring the defect hole closing material 100.

### DESCRIPTION OF EMBODIMENTS

Hereinafter, a medical material according to the present invention will be described in detail with reference to the drawings. While the following describes a defect hole closing material for use in catheterization as an example of the medical material according to the present invention, it is suitably applicable also to closure of another opening or passageway, for example, another opening in the heart due to a ventricular septal defect, patent ductus arteriosus, or the like, and an opening or a passageway at another site of a living body (for example, stomach), due to an arteriovenous fistula or the like. Accordingly, the defect hole closing material according to an embodiment of the present invention is not limited to be used for the closure of a hole arising from the atrial septal defect.

Moreover, although in the following embodiment, a stitch-like structure of a defect hole closing material (occluder) 100 will be described as an object obtained by knitting a bioabsorbable fiber (an example of a filamentary material), the present invention is not limited thereto. It is sufficient that the defect hole closing material enables catheterization adapted to close a defect hole formed in a living body, and therefore, its stitch-like structure (which does not include shape holding members as will be described later) may be knitted with a filamentary material other than the bioabsorbable fiber so long as the material has a first characteristic to a fourth characteristic as will be described later and exhibits a first action to a fourth action. Such a filamentary material preferably has a certain degree of hardness for the sake of shape retainability of the defect hole closing material (which differs from shape retainability by the shape holding members as will be described later).

### [Basic Configuration]

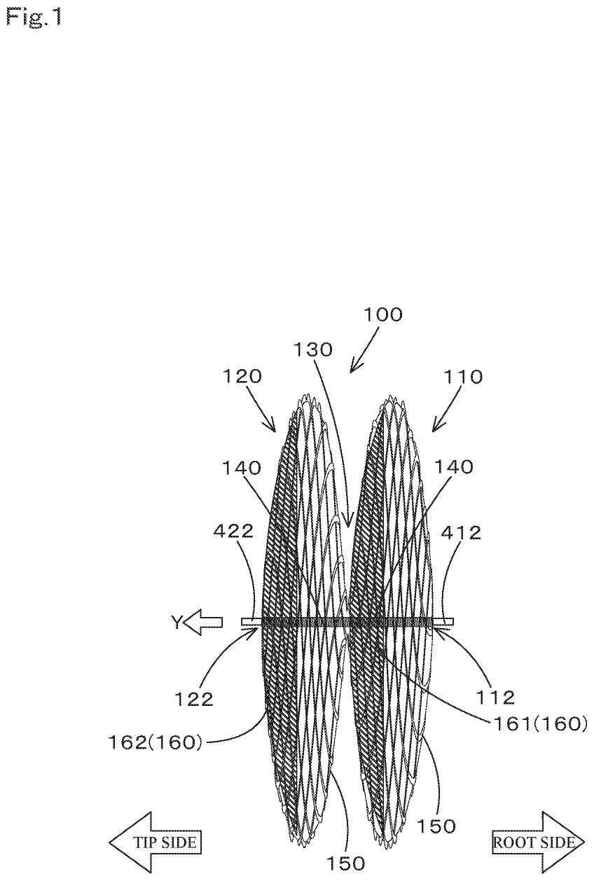

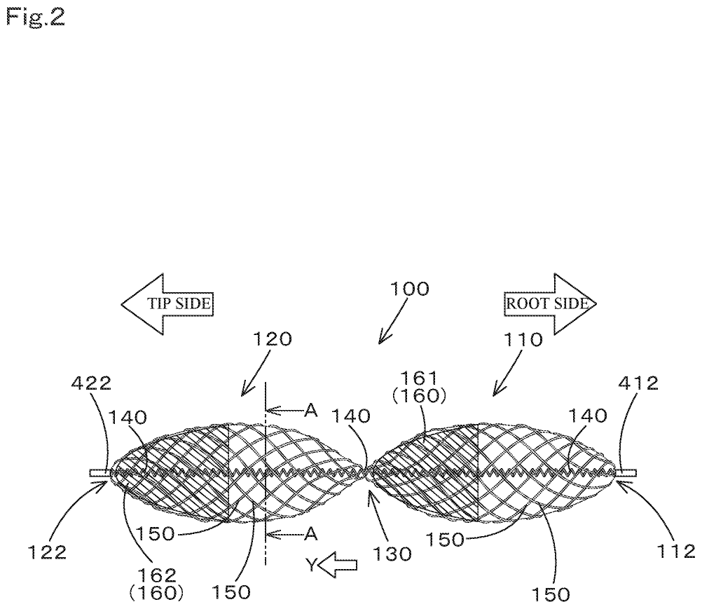

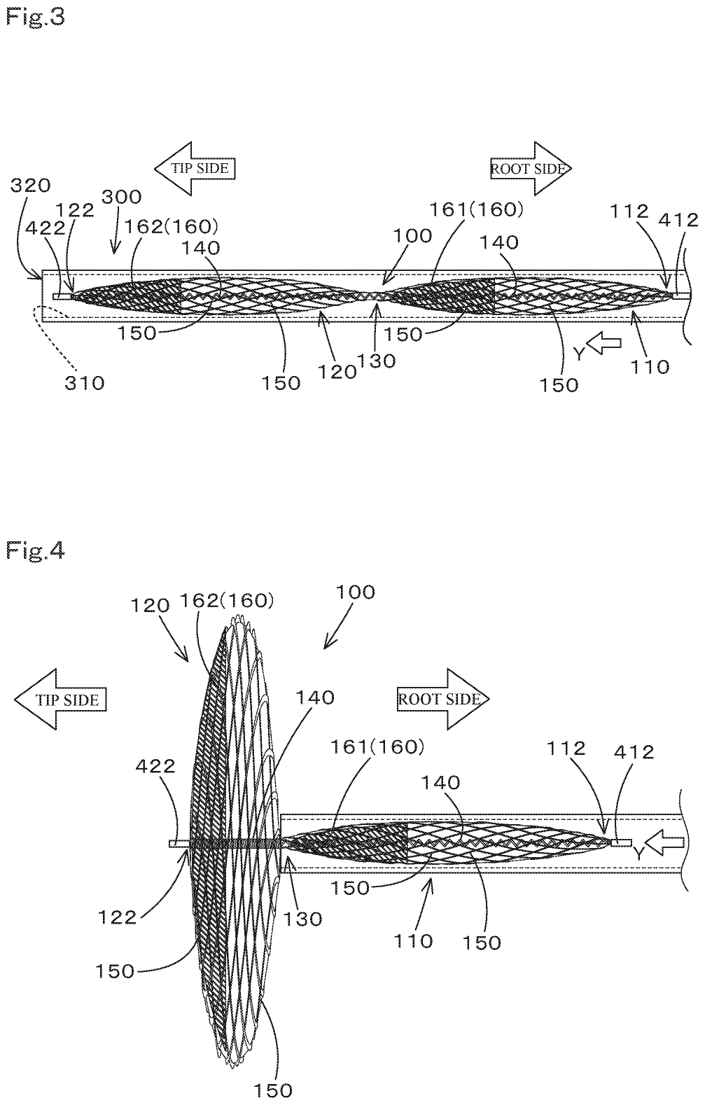

FIG. 1 is an overall view of the defect hole closing material 100 according to the embodiment (when a coil spring 140 is in a compressed state), FIG. 2 is an overall view of the defect hole closing material 100 (when the coil spring 140 is in an intermediate state), FIG. 3 is an overall view of the defect hole closing material 100 (when the coil spring 140 is in a stretched state), and FIG. 4 is an overall view of the defect hole closing material 100 (when the coil spring 140 is in the compressed state and in the stretched state). FIG. 3 is a view illustrating a state where the whole of the defect hole closing material 100 is contained in a catheter 300, and FIG. 4 is a view illustrating a state where half of the defect hole closing material 100 (a first cylinder part 110 side) is contained in the catheter 300. When pushing the defect hole closing material 100 that is wholly contained in the catheter 300 (in a space formed by an inner wall 310) illustrated in FIG. 3 from a first cylinder part 110 side in the direction indicated by an arrow Y to push out a second cylinder part 120 through an opening 320 of the catheter 300, the state of FIG. 4 is made. When further pushing out the first cylinder part 110 in the direction indicated by the arrow Y, the state of FIG. 1 is made. Note that the state of the defect hole closing material 100 illustrated in FIG. 2 is a virtual state where the coil spring 140 is in the intermediate state between the compressed state and the stretched state.

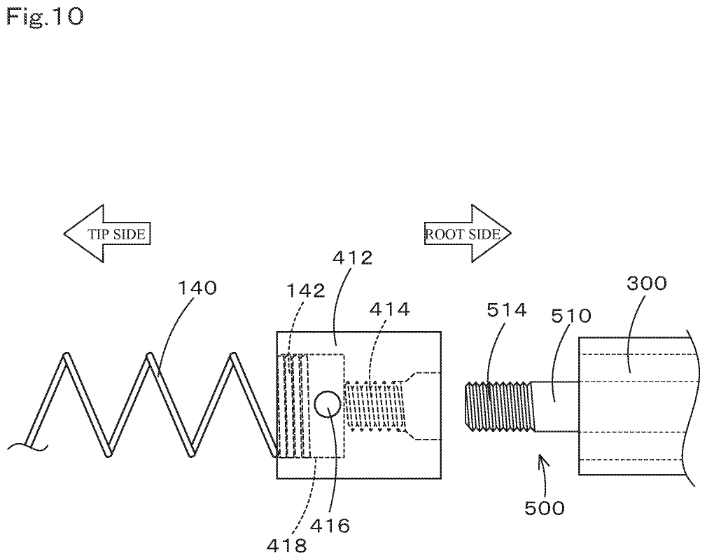

As will be described in detail later with reference to FIG. 10 to FIG. 16A and FIG. 16B, end parts of an elastic member (coil spring 140) in the defect hole closing material 100 are joined to small cylinder parts. The small cylinder parts are provided outside cylindrical bodies (the first cylinder part 110 and the second cylinder part 120) having the stitch-like structures, can be coupled to the shape holding members holding the shape of the defect hole closing material 100, and can be engaged with an operation wire 500. To be more specific, the small cylinder parts are a cylindrical metal piece 412 (root side) and a cylindrical metal piece 422 (tip side) and are formed such that both of the metal piece 412 on the root side and the metal piece 422 on the tip side can be coupled to the shape holding members and at least the metal piece 412 on the root side can be engaged with the operation wire 500.

As illustrated in these drawings, the defect hole closing material 100 is roughly formed by a cylindrical body having the stitch-like structure using the filamentary material and has such shape that the cylindrical diameter of a substantially central part 130 of the cylindrical body is smaller than the cylindrical diameters of the other parts, wherein the first cylinder part 110 on a first end part 112 side in the cylindrical body lengthwise direction of the defect hole closing material 100 and the second cylinder part 120 on the other end part (second end part 122) side are formed with the substantially central part 130 as a center. A characteristic point is to include, as an example of the elastic member, the coil spring 140 both ends of which are respectively engaged with the filamentary material at the first end part 112 and the filamentary material at the second end part 122 and that is inserted through inner parts of the first cylinder part 110 and the second cylinder part 120 from the first end part 112 side to the second end part 122 side via the substantially central part 130. Even in the case other than the coil spring 140, the elastic member is only required to be a member having elasticity and capable of exhibiting actions as will be described later with the elasticity and is not limited to the coil spring 140.

Another characteristic point is in that at least one of a first porous layer 161 arranged on the substantially central part 130 side in the first cylinder part 110 and a second porous layer 162 arranged on an opposite side to the substantially central part 130 in the second cylinder part 120 is arranged on an inner surface of the cylindrical body, the first porous layer 161 and the second porous layer 162 being formed by any one of a nonwoven fabric, sponge, film, and composite body of them. A material of the porous layers is not limited but the porous layers need to have such flexibility that the shapes of the porous layers can be changed along inner surface shapes of the cylindrical bodies (the first cylinder part 110 and the second cylinder part 120) as the other parts than the substantially central part 130 in the defect hole closing material 100 with increase/decrease in the cylinder diameters of the cylindrical bodies.

More specifically, the first porous layer 161 is arranged on the substantially central part 130 side in the lengthwise direction of the first cylinder part 110 and the second porous layer 162 is arranged on the opposite side to the substantially central part 130 in the lengthwise direction of the second cylinder part 120. Arrangement of the porous layers is not limited to the above-mentioned form, and it is sufficient that at least any one of the first porous layer 161 and the second porous layer 162 is arranged. Both of the first porous layer 161 and the second porous layer 162 may be arranged or either of the first porous layer 161 or the second porous layer 162 and another porous layer (for example, on the opposite side to the first porous layer 161 in the lengthwise direction in the first cylinder part 110 or on the opposite side to the second porous layer 162 in the lengthwise direction in the second cylinder part 120) may be arranged. Hereinafter, description is made while both of the first porous layer 161 and the second porous layer 162 are arranged at the above-mentioned positions (the first porous layer 161 is arranged on the substantially central part 130 side in the lengthwise direction, the second porous layer 162 is arranged on the opposite side to the substantially central part 130 in the lengthwise direction, and they are located at positions on the tip side in the lengthwise directions of the respective cylinder parts). Herein, the lengths of the first porous layer 161 and the second porous layer 162 in the lengthwise direction are substantially the same, and they are collectively referred to as porous layers 160 (in the following description and the drawings) in some cases.

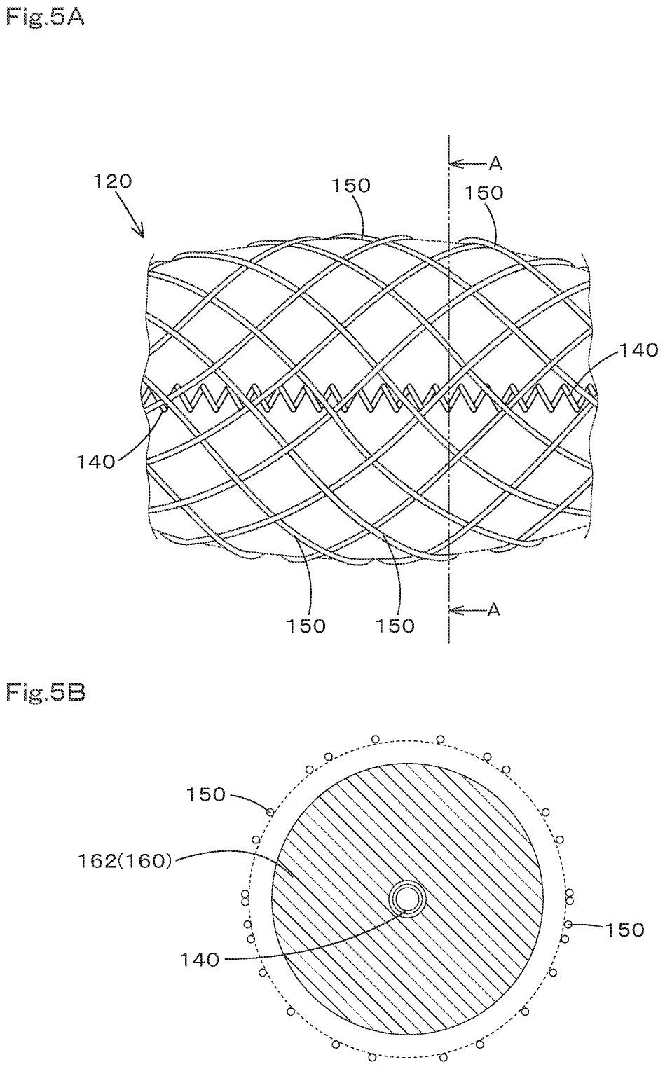

FIG. 5A is a partial side view of the defect hole closing material 100 and FIG. 5B is a cross-sectional view along A-A in FIG. 2 and FIG. 5A. Note that although FIG. 5B is the cross-sectional view of the defect hole closing material 100 (to be more specific, of the second cylinder part 120), FIG. 5B illustrates only the cross-sections of the coil spring 140 and a bioabsorbable fiber 150 and appearance of the second porous layer 162 and does not illustrate the stitches of the bioabsorbable fiber 150 viewable from a direction indicated by an arrow A. In addition, in FIG. 1 to FIG. 5, in order to facilitate the understanding of the presence of the coil spring 140 and the stitches of the bioabsorbable fiber 150, the bioabsorbable fiber 150 arranged on the deep side of the page is not illustrated, and in order to facilitate the understanding of an appearance shape of the defect hole closing material 100, the appearance shape of the defect hole closing material 100 is indicated by dotted line in some portions. In all of the drawings, the porous layers 160 (the first porous layer 161 and the second porous layer 162) arranged along the inner surfaces of the cylindrical bodies (the first cylinder part 110 and the second cylinder part 120) of the defect hole closing material 100 are indicated by applying hatching to the defect hole closing material 100.

As illustrated in these drawings (particularly in FIG. 2), the defect hole closing material 100 is formed by two cylindrical bodies (the first cylinder part 110 and the second cylinder part 120) made of the bioabsorbable material and having the stitch-like structures and has a shape formed by such two cylindrical bodies, which is called, for example, a sandglass shape, a figure-of-eight shape, a double spindle shape (the shape of two continuous elongated rod-like spindle-shaped objects whose middles are thick and both ends are thin), or a peanut shape (an appearance shape of a peanut shell containing two nuts). The defect hole closing material 100 having the above-mentioned shape has such a shape that the substantially central part 130 is narrowed so as to make the cylinder diameter of the substantially central part 130 smaller than the cylinder diameters of the other parts. That is, the first cylinder part 110 on the first end part 112 side and the second cylinder part 120 on the second end part 122 side are formed with the substantially central part 130 as the center.

Although not limited, in the defect hole closing material 100, the first cylinder part 110 and the second cylinder part 120 are integrally knitted such that the cylinder diameter of the substantially central part 130 is made smaller than the cylinder diameters of the other parts, and the defect hole closing material 100 is formed into, as the whole shape, the sandglass shape, figure-of-eight shape, double spindle shape, or peanut shape formed by the two cylindrical bodies.

As is understood from FIG. 2, the first porous layer 161 and the second porous layer 162 (the porous layers 160 provided at two places) have umbrella shapes along the sandglass shape, figure-of-eight shape, double spindle shape, or peanut shape. The porous layers have flexibility, and the shapes thereof also change with change in the shapes of the cylindrical bodies as described above. The shapes matching with the shapes of the cylindrical bodies (the first cylinder part 110 and the second cylinder part 120) of the defect hole closing material 100 when the coil spring 140 is in the intermediate state illustrated in FIG. 2 are therefore the umbrella shapes as representative shapes of the porous layers. The porous layers 160 are however not limited to have complete umbrella shapes due to a material characteristic that the porous layers 160 are formed by any one of the nonwoven fabric, sponge, film, and composite body of them, and are not limited to be arranged completely along the inner surfaces of the cylindrical bodies (the first cylinder part 110 and the second cylinder part 120) of the defect hole closing material 100.

In this case, the whole shape of the defect hole closing material 100 is formed by using a frame (a three-dimensional paper pattern) of such a sandglass shape, figure-of-eight shape, double spindle shape, or peanut shape to knit one bioabsorbable fiber 150 in conformity with the frame. Further, although not limited, the defect hole closing material 100 may be formed into the sandglass shape, figure-of-eight shape, double spindle shape, or peanut shape formed by the two cylindrical bodies as the whole shape of the defect hole closing material 100 by integrally knitting the first cylinder part 110 and the second cylinder part 120 to knit a cylindrical body having a substantially uniform diameter, and then, thermally setting it and so on to thereby form the substantially central part 130 having the cylinder diameter that is smaller than the cylinder diameters of the other parts and is larger than the diameter of the coil spring 140. As will be described in detail later, the above-mentioned shape makes it possible to achieve the following change in shape. That is, when pushing the defect hole closing material 100 that is wholly contained in the catheter 300 (in the space formed by the inner wall 310) illustrated in FIG. 3 from the first cylinder part 110 side in the direction indicated by the arrow Y to push out the second cylinder part 120 through the opening 320 of the catheter 300, the second cylinder part 120 is released from the space formed by the inner wall 310 of the catheter 300 to compress the coil spring 140 in the second cylinder part 120, and the state of FIG. 4 is made. When further pushing out the first cylinder part 110 in the direction indicated by the arrow Y, the first cylinder part 110 is released from the space formed by the inner wall 310 of the catheter 300 to compress the coil spring 140 in the first cylinder part 110, and the state of FIG. 1 is made.

Furthermore, the defect hole closing material 100 includes the coil spring 140 one end of which is coupled to the cylindrical metal piece 412 provided on the first end part 112 side, the other end of which is coupled to the cylindrical metal piece 422 provided on the second end part 122 side, and that is inserted through the inner parts of the first cylinder part 110 and the second cylinder part 120 from the first end part 112 side to the second end part 122 side via the substantially central part 130. Coupling between the coil spring 140 and the cylindrical metal piece 412 and the cylindrical metal piece 422 will be described later.

As illustrated in FIG. 1, when the coil spring 140 is in the compressed state, the first end part 112 and the second end part come close to each other with the substantially central part 130 as the center, and the cylinder diameters of the first cylinder part 110 and the second cylinder part 120 as the other parts than the substantially central part 130 are increased. Particularly preferably, when the coil spring 140 is in the compressed state, the cylinder diameters of the first cylinder part 110 and the second cylinder part 120 as the other parts than the substantially central part 130 are increased to sizes corresponding to a defect hole to be closed by the defect hole closing material 100. The first porous layer 161 and the second porous layer 162 expand to sizes corresponding to the defect hole to be closed by the defect hole closing material 100 with the increase in the cylinder diameters of the first cylinder part 110 and the second cylinder part 120 as the other parts than the substantially central part 130.

As illustrated in FIG. 3, when the coil spring 140 is brought into the stretched state by containing the defect hole closing material 100 in the catheter 300 and so on, the first end part 112 and the second end part 122 are separated from each other with the substantially central part 130 as the center, and the cylinder diameters of the first cylinder part 110 and the second cylinder part 120 as the other parts are decreased. Particularly preferably, when the coil spring 140 is in the stretched state, the cylinder diameters of the first cylinder part 110 and the second cylinder part 120 as the other parts are decreased to sizes corresponding to the catheter 300 in which the defect hole closing material 100 is to be contained. The first porous layer 161 and the second porous layer 162 contract to sizes corresponding to the catheter 300 in which the defect hole closing material 100 is to be contained with the decrease in the cylinder diameters of the first cylinder part 110 and the second cylinder part 120 as the other parts.

As described above, by using the coil spring 140 having the diameter smaller than the cylinder diameter of the substantially central part 130, the first end part 112 and the second end part 122 as the other end part in the lengthwise direction of the cylindrical bodies in the defect hole closing material 100 can be brought close to or separated from each other. When the coil spring 140 is made into the compressed state, as illustrated in FIG. 1, the first end part 112 and the second end part 122 come close to each other to increase the cylinder diameters of the other parts than the substantially central part 130 (the cylinder diameters of body parts of the first cylinder part 110 and the second cylinder part 120). When the coil spring 140 is made into the stretched state, as illustrated in FIG. 3, the first end part 112 and the second end part 122 are separated from each other to decrease the cylinder diameters of the other parts than the substantially central part 130 (the cylinder diameters of the body parts of the first cylinder part 110 and the second cylinder part 120). Further, as illustrated in FIG. 4, when pushing the second cylinder part 120 out of the catheter 300 in the direction indicated by the arrow Y, the second cylinder part 120 the shape of which has been restricted by the inner wall 310 of the catheter 300 can freely change its shape, and only the part of the coil spring 140 that is contained in the second cylinder part 120 is compressed to increase only the cylinder diameter of the body part of the second cylinder part 120. When further pushing the first cylinder part 110 out of the catheter 300 in the direction indicated by the arrow Y, the first cylinder part 110 the shape of which has been restricted by the inner wall 310 of the catheter 300 can also freely change its shape, and the part of the coil spring 140 that is contained in the first cylinder part 110 is also compressed to increase the cylinder diameter of the body part of the first cylinder part 110 as well, as illustrated in FIG. 1.

In the defect hole closing material 100, the porous layers 160 (the first porous layer 161 and the second porous layer 162) formed by any one of the nonwoven fabric, sponge, film, and composite body of them are arranged on the inner surfaces of the cylindrical bodies. The first cylinder part 110 and the second cylinder part 120 are formed by a woven fabric (open one), knitted fabric, braid-like fabric, or cylindrically-knitted fabric of the bioabsorbable fiber 150 and are wholly formed as the stitch-like structures. As is described here for confirmation, the stitch-like structure includes, without limitation to the knitted fabric formed by knitting, a mesh-like structure formed as an open woven structure like a screen door as described above. That is, the structure called “stitch-like” and the structure called “mesh-like” are both applicable. The porous layers 160 (the first porous layer 161 and the second porous layer 162) are formed by any one of the non-woven fabric, sponge, film, and composite body of them based on the assumption that it holds a medical agent by application, impregnation, embedding, or the like. Further, the porous layers 160 (the first porous layer 161 and the second porous layer 162) are not limited to be made of the bioabsorbable material but may be made of a non-bioabsorbable material. The porous layers 160 (the first porous layer 161 and the second porous layer 162) have flexibility as described above. The porous layers 160 (the first porous layer 161 and the second porous layer 162) arranged on the inner surfaces of the first cylinder part 110 and the second cylinder part 120 therefore also expand/contract (by being changed in shapes similarly to the change in the shapes of the cylindrical bodies) with the increase/decrease in the cylinder diameters of the cylindrical bodies (the first cylinder part 110 and the second cylinder part 120) as the other parts than the substantially central part 130.

As described above, basically except for the coil spring 140 made of a nickel-titanium alloy, for example, the metal piece 412 and the metal piece 422 made of stainless steel, for example, and the shape holding members (a first shape holding member 610 on the root side and a second shape holding member 620 on the tip side) made of a surgical suture, for example, the first cylinder part 110, the second cylinder part 120, and the porous layers 160 (the first porous layer 161 and the second porous layer 162) are all made of the bioabsorbable material. Accordingly, the whole of the defect hole closing material 100 except for the coil spring 140, the metal piece 412, and the metal piece 422 has bioabsorbability (the surgical suture forming the first shape holding member 610 and the second shape holding member 620 preferably has the bioabsorbability in order to improve bioabsorbability). In addition, the change in the shape of the defect hole closing material 100 allows the treatment for closing the defect hole to be performed, and the defect hole closing material 100 including the porous layers 160 (the first porous layer 161 and the second porous layer 162) is formed while employing a material, stitch shape, fiber structure, and fiber cross section that prevent an in vivo tissue from being damaged even when the shape of the defect hole closing material 100 is thus changed in the living body.

Normally, the coil spring 140 is made of, for example, the nickel-titanium alloy and does not have the bioabsorbability, and the metal piece 412 and the metal piece 422 are made of, for example, the stainless steel and does not have the bioabsorbability. Alternatively, they may be made of a magnesium-based alloy as will be described later to have the bioabsorbability. Usage of an alloy having the bioabsorbability for the coil spring 140, the metal piece 412, and the metal piece 422 is advantageous in terms of reacting to X-ray imaging, and usage of the alloy having the bioabsorbability is advantageous in terms of preventing the problem of fear of long-term failure because no metallic member remains in the body through the whole life.

The bioabsorbable fiber 150 forming the first cylinder part 110 and the second cylinder part 120 is, for example, of at least one type selected from polyglycolic acid, polylactide (D, L, and DL isomers), polycaprolactone, glycolic acid-lactide (D, L, and DL isomers) copolymers, a glycolic acid-ε-caprolactone copolymer, lactide (D, L, and DL isomers)-ε-caprolactone copolymers, poly (p-dioxanone), glycolic acid-lactide (D, L, and DL isomers)-ε-caprolactone copolymers, and the like. While the selected material is used after being processed into any one of a monofilament yarn form, multifilament yarn form, twisted yarn form, braid form, and the like, the material is preferably used in the monofilament yarn form.

Further, the material of the bioabsorbable fiber 150 may be a bioabsorbable alloy. Examples of such a bioabsorbable alloy include the alloy based on magnesium as a raw material.

The bioabsorbable fiber 150 is designed to have a diameter in a range of about 0.001 mm to 1.5 mm, and a fiber diameter and type suitable for catheterization to be applied are selected. Also, the cross section of the bioabsorbable fiber 150 may be any one of a circle, ellipse, and other different shapes (such as a star shape) under the condition that the in vivo tissue is not damaged. Further, the surface of the bioabsorbable fiber 150 may be subjected to hydrophilic treatment by plasma discharge, electron beam treatment, corona discharge, ultraviolet irradiation, ozone treatment, or the like. Moreover, the bioabsorbable fiber 150 may be subjected to application or impregnation processing with an X-ray non-permeable material (such as barium sulfide, a gold chip, and a platinum chip), adhesion processing of a medical agent (such as a medical agent suitable for catheterization for an atrial septal defect), or coating processing with a natural polymer such as collagen and gelatin or a synthetic polymer such as polyvinyl alcohol and polyethylene glycol.

The first cylinder part 110 and the second cylinder part 120 are formed as follows. That is, the bioabsorbable fiber 150 is knitted into a braid-like textile using a braiding machine with multiple (for example, 8 or 12) yarn feeders around a silicone-made rubber tube (not illustrated) having an outer diameter desired as the monofilament yarn, for example, or knitted into a cylindrical stitch-like structure having a substantially uniform diameter using a circular knitting machine (not illustrated). After the knitting, as described above, the braid-like textile or the cylindrical stitch-like structure is formed into the sandglass shape, figure-of-eight shape, double spindle shape, or peanut shape formed by the two cylindrical bodies of the first cylinder part 110 and the second cylinder part 120. The cylinder diameters of the first cylinder part 110 and the second cylinder part 120 are set to be smaller than the inner diameter of the catheter in the diameter-decreased states and to those preferable for the catheterization for the atrial septal defect in the diameter-increased states. For example, the cylinder diameters of the first cylinder part 110 and the second cylinder part 120 in the diameter-increased states are in a range of 5 mm to 80 mm, and preferably in a range of about 15 mm to 25 mm. In addition, the lengths of the first cylinder part 110 and the second cylinder part 120 and the density of the stitch-like structure of the defect hole closing material 100 are also set to be sizes preferable for the catheterization for the atrial septal defect. Note that the cylinder diameters and lengths of the first cylinder part 110 and the second cylinder part 120 do not have to be the same but may be changed so as to be preferable for the catheterization for the atrial septal defect.

The bioabsorbable material forming the porous layers 160 (the first porous layer 161 and the second porous layer 162) is not particularly limited, and examples thereof include synthetic absorbable polymers such as polyglycolic acid, polylactide (D, L, and DL isomers), polycaprolactone, glycolic acid-lactide (D, L, and DL isomers) copolymers, a glycolic acid-ε-caprolactone copolymer, lactide (D, L, and DL isomers)-ε-caprolactone copolymers, poly (p-dioxanone), and glycolic acid-lactide (D, L, and DL isomers)-ε-caprolactone copolymers. These may be used individually, or equal to or more than two types thereof may be used in combination. Among them, at least one type selected from a group consisting of polyglycolic acid, lactide (D, L, and DL isomers)-ε-caprolactone copolymers, a glycolic acid-ε-caprolactone copolymer, and glycolic acid-lactide (D, L, and DL isomers)-ε-caprolactone copolymers is preferable because they exhibit appropriate degradation behaviors, and the porous layers 160 are formed by any one of the non-woven fabric, sponge, film, and composite body of them. In particular, as a preferred mode, the non-woven fabric can be exemplified.

Further, the material of the porous layers 160 (the first porous layer 161 and the second porous layer 162) may be the bioabsorbable alloy. Examples of such a bioabsorbable alloy include an alloy based on magnesium as a raw material.

When the porous layers 160 (the first porous layer 161 and the second porous layer 162) are formed by the non-woven fabric, hydrophilic treatment may be applied. The hydrophilic treatment is not particularly limited, and for example, plasma treatment, glow discharge treatment, corona discharge treatment, ozone treatment, surface grafting treatment, ultraviolet irradiation treatment, or the like can be performed. Among them, the plasma treatment is preferable because the plasma treatment can dramatically improve a water absorption rate without changing the appearance of the non-woven fabric layer. Note that each of the porous layers 160 (the first porous layer 161 and the second porous layer 162) may be a sponge layer, a film layer, a composite layer of a non-woven fabric and the sponge layer, a composite layer of the non-woven fabric and the film layer, a composite layer of the sponge layer and the film layer, or a composite layer of the non-woven fabric, the sponge layer, and the film layer.

The porous layers 160 (the first porous layer 161 and the second porous layer 162) preferably hold the medical agent suitable for the catheterization for the atrial septal defect.

As described above, the defect hole closing material 100 according to the embodiment has the following characteristics.

(First Characteristic) The defect hole closing material 100 is formed into the sandglass shape, figure-of-eight shape, double spindle shape, or peanut shape narrowed in the substantially central part 130 and formed by the first cylinder part 110 and the second cylinder part 120.
(Second Characteristic) The defect hole closing material 100 includes the coil spring 140 one end of which is engaged with the first end part 112 (caught on the looped filamentary material at the first end part 112), the other end of which is engaged with the second end part 122 (caught on the looped filamentary material at the second end 122), and that is inserted through the inner parts of the first cylinder part 110 and the second cylinder part 120 from the first end part 112 side to the second end part 122 side via the substantially central part 130.
(Third Characteristic) The defect hole closing material 100 is formed by the first cylinder part 110, the second cylinder part 120, the coil spring 140 (when made of the magnesium-based alloy), and the porous layers 160 (the first porous layer 161 and the second porous layer 162), and these components are all made of the bioabsorbable material (the coil spring 140 does not necessarily have the bioabsorbability).
(Fourth characteristic) The first porous layer 161 is arranged on the substantially central part 130 side in the lengthwise direction of the first cylinder part 110 and the second porous layer 162 is arranged on the opposite side to the substantially central part 130 in the lengthwise direction of the second cylinder part 120 such that they are arranged on the inner surfaces of the respective cylindrical bodies so as to be along the inner surfaces.

With the first characteristic and the second characteristic, in the defect hole closing material 100 contained in the catheter 300, when pushing the second cylinder part 120 out of the catheter 300, the second cylinder part 120 the shape of which has been restricted by the inner wall 310 of the catheter 300 can freely change its shape, and only the part of the whole of the coil spring 140 that is contained in the second cylinder part 120 can be compressed to increase only the cylinder diameter of the body part of the second cylinder part 120, and when further pushing the first cylinder part 110 out of the catheter 300, the first cylinder part 110 the shape of which has been restricted by the inner wall 310 of the catheter 300 can also freely change its shape, and the part of the whole of the coil spring 140 that is contained in the first cylinder part 110 can also be compressed to increase the cylinder diameter of the body part of the first cylinder part 110 as well. The first porous layer 161 and the second porous layer 162 expand to the sizes corresponding to the defect hole to be closed by the defect hole closing material 100 with the above-mentioned increase in the cylinder diameters of the body parts.

In particular, the defect hole closing material 100 is suitable for the catheterization for the atrial septal defect because it provides the following actions.

(First Action) The defect hole closing material 100 can be set in the catheter 300 by stretching the whole of the coil spring 140 to make the cylinder diameter of the defect hole closing material 100 including the porous layers smaller than the inner diameter of the catheter 300.
(Second Action) The defect hole closing material 100 is set in the catheter 300 and sent to a position of a hole opening in the atrial septum. Then, when pushing the first end part 112 with an applicator or the like in a living body to push the second cylinder part 120 out of the catheter 300 into the living body, the coil spring 140 in the second cylinder part 120 is compressed to increase the cylinder diameter of the body part of the second cylinder part 120, and the second porous layer 162 expands to the size corresponding to the defect hole to be closed by the defect hole closing material 100. When further pushing the first end part 112 with the applicator or the like to push the first cylinder part 110 out of the catheter 300 into the living body, the coil spring 140 in the first cylinder part 110 is also compressed to increase the cylinder diameter of the body part of the first cylinder part 110 as well, and the first porous layer 161 expands to the size corresponding to the defect hole to be closed by the defect hole closing material 100. Moreover, the first cylinder part 110 arranged on the right atrium side and the second cylinder part 120 arranged on the left atrium side come close to each other with the substantially central part 130 as the center to occlude the hole opening in the atrial septum.
(Third Action) The components (excluding the coil spring 140, the metal piece 412, and the metal piece 422 in some cases) forming the defect hole closing material 100 are all made of the bioabsorbable material, so that they are finally absorbed in the living body and fear of long-term failure is almost eliminated.
(Fourth Action) The first porous layer 161 and the second porous layer 162 included in the defect hole closing material 100 have the umbrella shapes tapered toward the traveling direction (tip side as the direction indicated by the arrow Y) (are not enlarged toward the traveling direction), so that resistance in pushing in the direction indicated by the arrow Y is low. The first cylinder part 110 and the second cylinder part 120 and the first porous layer 161 and the second porous layer 162 can accurately occlude the hole opening in the atrial septum.

### [Shape Holding Member]

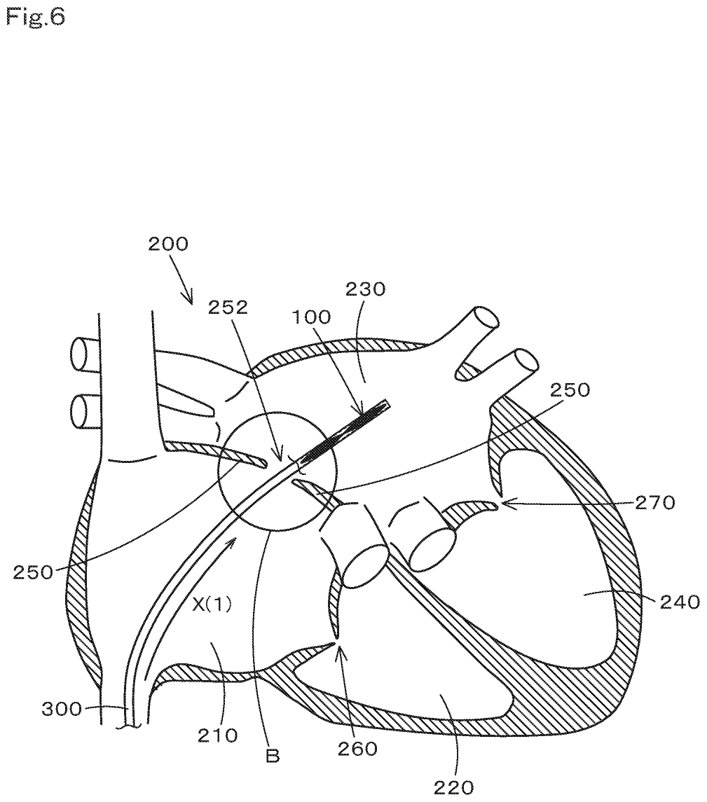

As will be described in detail later as a usage mode, when the defect hole closing material 100 is used for the catheterization for the atrial septal defect, as illustrated in FIG. 6 to FIG. 9, for example, the defect hole closing material 100 is set to the catheter 300 by making the cylinder diameter of the defect hole closing material 100 smaller than the inner diameter of the catheter 300. The catheter 300 containing the defect hole closing material 100 is then inserted from the femoral vein to bring the catheter 300 containing the defect hole closing material 100 close to a left atrium 230 side through a defect hole 252 from a right atrium 210. The first cylinder part 110 and the second cylinder part 120 are pushed in this order out of the catheter 300 in the direction indicated by the arrow Y with the applicator or the like (the operation wire 500 as an example). In this case, force of pushing them out of the catheter 300 with the operation wire 500 along the lengthwise direction for moving the defect hole closing material 100 along the lengthwise direction acts on the defect hole closing material 100.

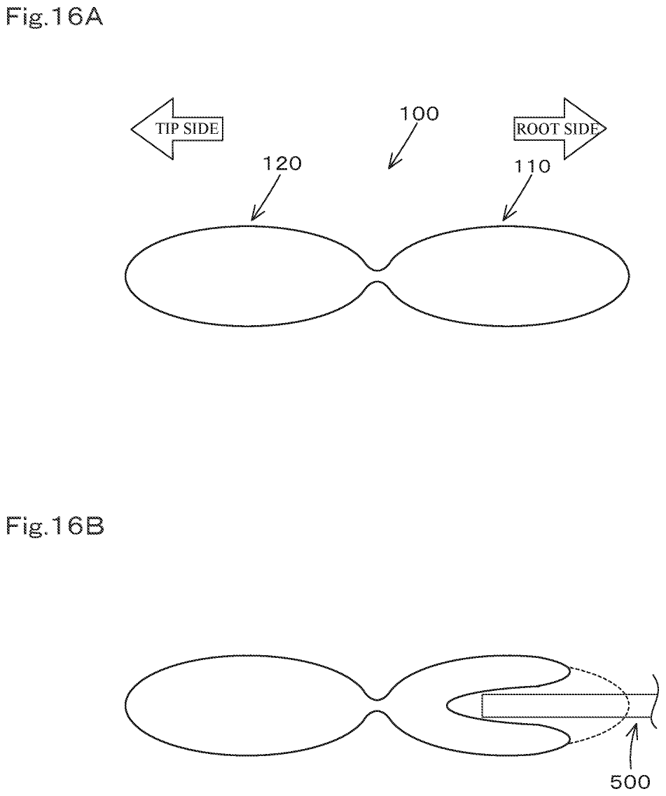

When the push-out force with the operation wire 500 acts on the defect hole closing material 100 in a state illustrated in FIG. 16A, the shape of the defect hole closing material 100 cannot be held in some cases as illustrated in FIG. 16B. That is to say, the filamentary material 150 of the first cylinder part 110 is deformed and the shape of the defect hole closing material 100 is changed in some cases as illustrated in FIG. 16B because the push-out force with the operation wire 500 can act only from the root side of the defect hole closing material 100. To cope with this, the defect hole closing material 100 in the embodiment includes the shape holding member as will be described in detail below as a remarkable characteristic.

The shape holding member is at least any one of the first shape holding member 610 on the first cylinder part 110 side (root side) of the defect hole closing material 100 and the second shape holding member 620 on the second cylinder part 120 side (tip side) of the defect hole closing material 100 and is provided at at least any one of both ends of the coil spring 140 (hereinafter, both of the first shape holding member 610 and the second shape holding member 620 are included for description), and they are coupled to the end parts of the coil spring 140. The shape holding members hold the shape of the defect hole closing material 100 against external force (although it indicates the push-out force with the operation wire 500 herein, it is simply referred to as external force) acting along the lengthwise direction of the defect hole closing material 100 for moving the defect hole closing material 100 (itself).

The end parts of the coil spring 140 to which the shape holding members are coupled are joined to the small cylinder parts provided outside the cylindrical bodies having the stitch-like structures and capable of being engaged with the operation wire, so that the small cylinder parts are integrated with the coil spring 140. The shape holding members are coupled to the small cylinder parts integrated with the coil spring 140. To be more specific, as described above, the small cylinder parts are the cylindrical metal piece 412 (root side) and the cylindrical metal piece 422 (tip side) and are formed such that both of the metal piece 412 on the root side and the metal piece 422 on the tip side can be coupled to the shape holding members and at least the metal piece 412 on the root side can be engaged with the operation wire 500.

The defect hole closing material 100 in the embodiment includes the shape holding members as the remarkable characteristic as described above. The cylindrical metal piece 412 and the cylindrical metal piece 422 as the small cylinder parts are however described first because the shape holding members are coupled to the metal piece 412 and the metal piece 422.

In the defect hole closing material 100, the end parts of the elastic member (coil spring 140) are joined to the small cylinder parts provided outside the cylindrical bodies (the first cylinder part 110 and the second cylinder part 120) having the stitch-like structures and capable of being engaged with the operation wire 500.

The cylindrical metal piece 412 and the cylindrical metal piece 422 as the small cylinder parts to which the shape holding members are coupled will be described with reference to a partial enlarged view of the root side in FIG. 1 and FIG. 2, which is illustrated in FIG. 10, in addition to the overall view of the defect hole closing material 100 (when the coil spring 140 is in the compressed state) illustrated in FIG. 1 and the overall view of the defect hole closing material 100 (when the coil spring 140 is in the intermediate state) illustrated in FIG. 2. The following describes, of the metal piece 412 and the metal piece 422, the metal piece 412 on the root side that is formed to be capable of being engaged with the operation wire 500. The metal piece 422 on the tip side may be formed or may not be formed to be capable of being engaged with the operation wire 500, and it is assumed that the metal piece 422 on the tip side is not formed to be capable of being engaged with the operation wire 500 below. The configuration and action effect of the metal piece 422 on the tip side are the same as those of the metal piece 412 on the root side other than the point that it is not formed to be capable of being engaged with the operation wire 500 (in particular, a joint form with the coil spring 140 and a coupling form to the shape holding member). The metal piece 412 on the root side is therefore described as a representative in some cases.

As illustrated in these drawings, the metal piece 412 on the root side has a female screw part 414 screwable with a male screw part 514 provided on a tip part 510 of the operation wire 500 that is inserted into the catheter 300. The metal piece 422 on the tip side and the metal piece 412 on the root side have the same configuration other than this point.

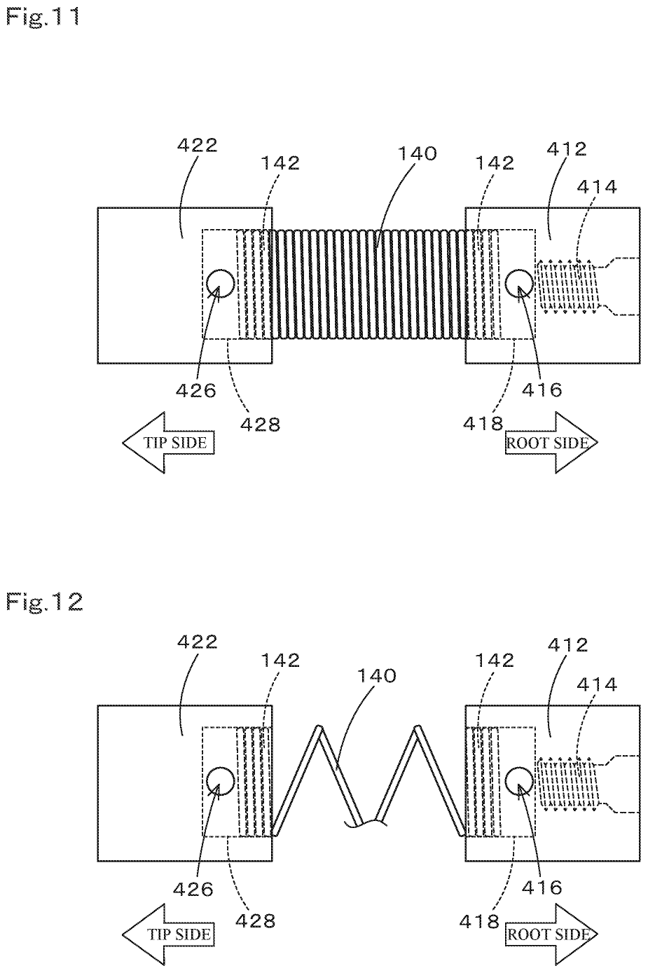

As illustrated in these drawings, the metal piece 422 on the tip side and the metal piece 412 on the root side are provided outside the cylindrical bodies (the first cylinder part 110 and the second cylinder part 120) having the stitch-like structures. Both end parts 142 of the coil spring 140 are joined to the small cylinder parts (to be more specific, the metal piece 422 on the tip side and the metal piece 412 on the root side) that are inserted into the catheter 300. For example, as illustrated in FIG. 10 (in this example, the metal piece 412 on the root side represents the metal piece 412 on the root side and the metal piece 422 on the tip side), the coil spring 140 having the both end parts 142 one of which has been joined to the metal piece 412 is inserted through the inner parts of the first cylinder part 110 and the second cylinder part 120 from the first end part 112 side to the second end part 122 side via the substantially central part 130. The small cylinder parts may be made of a material other than metal. It can be exemplified that the nickel-titanium alloy is used for the coil spring 140 and the stainless steel is used for the metal piece 410 as described above. As a joining method in the case of the combination of such metals, joining by caulking can be exemplified while a caulking hole 418 having a slightly smaller diameter than the diameters of the both end parts 142 of the coil spring 140 is provided in the metal piece 412 having a small cylindrical shape as illustrated in FIG. 10. The joint between the coil spring 140 and the metal piece 412 and the metal piece 422 thus joined to the coil spring 140 by caulking is not released even when the coil spring 140 is stretched and compressed, as illustrated in FIG. 11 and FIG. 12.

One end of the coil spring 140 is engaged with the first end part 112 (for example, caught on the loop of the filamentary material at the first end part 112) and the other end is engaged with the second end part 122 (for example, caught on the loop of the filamentary material at the second end part 122). The coil spring 140 having the both end parts 142 joined to the metal piece 412 and the metal piece 422 is inserted through the inner parts of the first cylinder part 110 and the second cylinder part 120 from the first end part 112 side to the second end part 122 side via the substantially central part 130.

As described above, engagement between both ends of the coil spring 140 and the filamentary material at the first end part 112 and the filamentary material at the second end part 122 includes direct engagement between the coil spring 140 and the filamentary material at both ends of the defect hole closing material 100 and engagement between the coil spring 140 and the filamentary material at both ends of the defect hole closing material 100 with the metal piece 412 and the metal piece 422 interposed therebetween. That is to say, the engagement includes the case in which the metal piece 412 and the metal piece 422 are joined to the coil spring 140 by caulking or the like, and the metal piece 412 and the metal piece 422 are joined to the filamentary material at both ends of the defect hole closing material 100, as described above.

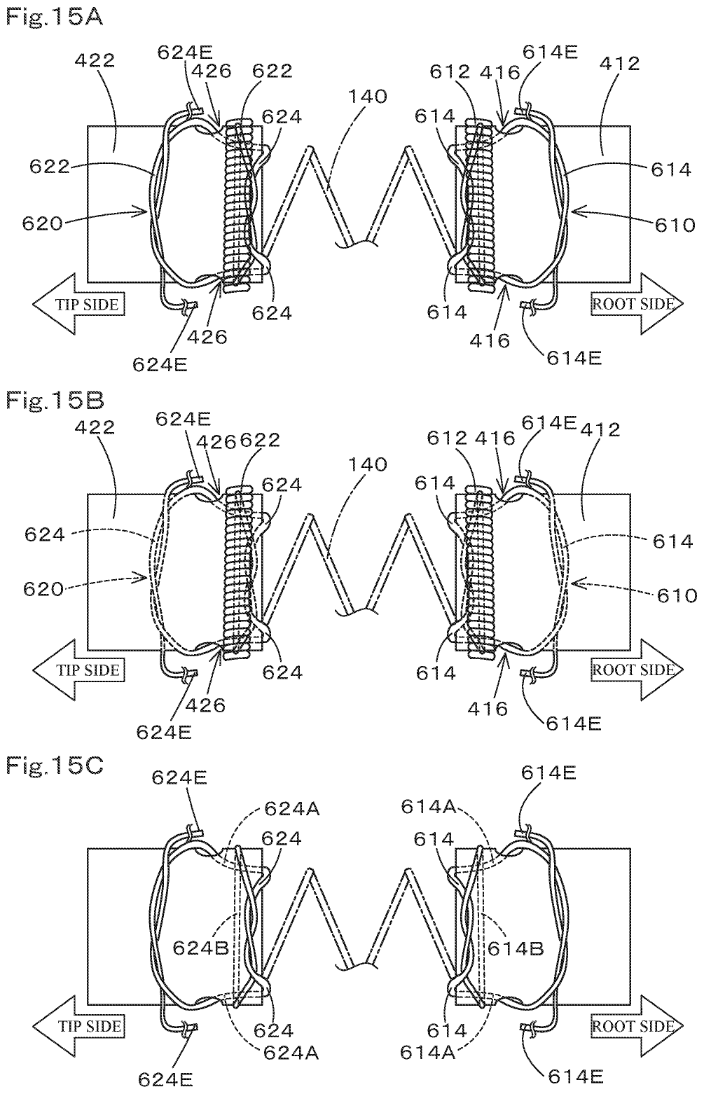

For coupling to the shape holding members, as illustrated in FIG. 10 to FIG. 12, the metal piece 412 and the metal piece 422 respectively have two through-holes 416 and two through-holes 426 penetrating from the outer surfaces to the inner surfaces of the small cylindrical shapes in the outer surfaces of the metal piece 412 and the metal piece 422 so as to be separated from each other by approximately 180 degrees (as illustrated in FIG. 15A to FIG. 15C). The above-mentioned caulking holes and the through-holes are coupled to each other (the caulking holes and the through-holes are provided so as to penetrate from the outer surfaces of the metal pieces having hollow cylindrical shapes to the cylinder inner sides). The insertion yarns can therefore be inserted through to the outer surface sides of the metal pieces through the through-holes while passing through the cylinder inner sides of the small cylindrical shapes from the caulking hole sides of the metal pieces.

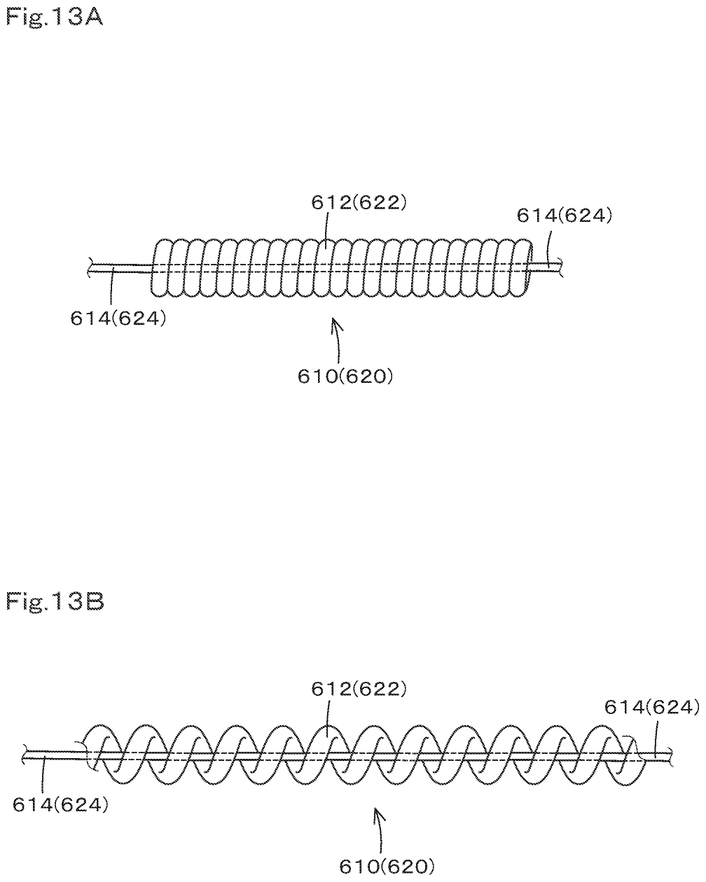

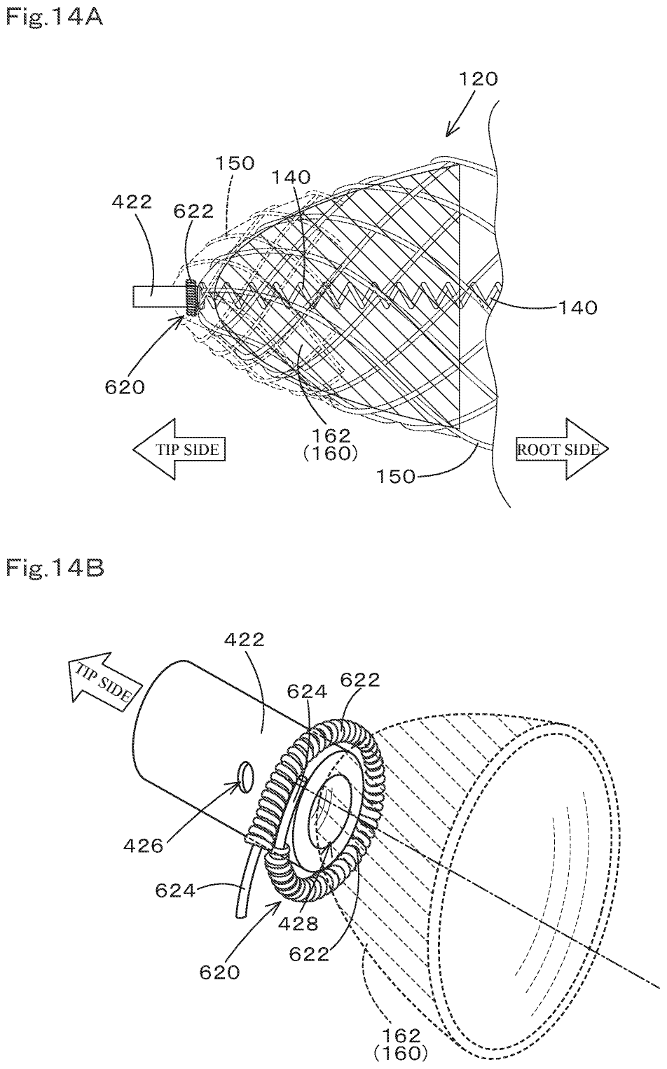

The shape holding members include, for example, the first shape holding member 610 on the first cylinder part 110 side (root side) of the defect hole closing material 100 and the second shape holding member 620 on the second cylinder part 120 side (tip side) of the defect hole closing material 100 that are provided at both ends of the coil spring 140 as illustrated in FIG. 14A and FIG. 14B and FIG. 15A to FIG. 15C (in FIG. 14A and FIG. 14B, the second shape holding member 620 represents the first shape holding member 610 and the second shape holding member 620). (Main bodies of) The shape holding member 610 and the shape holding member 620 include an exterior part 612 and an exterior part 622 formed by winding yarns (hereinafter, also referred to as exterior yarns in some cases) into spiral shapes and an insertion yarn 614 and an insertion yarn 624 inserted through the centers of the spiral shapes of the exterior part 612 and the exterior part 622, respectively, as illustrated in FIG. 13A and FIG. 13B. The state in FIG. 13A indicates a state in which no force acts on the exterior part and the state in FIG. 13B indicates a state in which force acts on the exterior part in the right-left direction of the page and the exterior part having the spiral shape (curl shape) is stretched. In both of the cases, the exterior part holds the spiral (curl) shape, and the shape is preferable because the exterior part effectively absorbs the external force.

When the first shape holding member 610 and the second shape holding member 620 are thus provided at both ends of the coil spring 140, the thickness of the exterior yarn forming the exterior part 612 and the thickness of the exterior yarn forming the exterior part 622 differ from each other. In particular, it is preferable that when the defect hole closing material 100 is contained in the catheter 300, the yarn forming the exterior part be thicker in the shape holding member on the root side than the shape holding member on the tip side. That is to say, it is particularly preferable that the thickness of the exterior yarn of the exterior part 612 in the first shape holding member 610 on the root side be larger than the thickness of the exterior yarn of the exterior part 622 in the second shape holding member 620 on the tip side.

More specifically, although not limited, a surgical suture is used for both of the exterior yarn and the insertion yarn. The thickness of the yarn (the diameter of the surgical suture) is as follows:

the thickness of the exterior yarn of the exterior part 612 in the first shape holding member 610 on the root side as the thicker one is USP size 3-0 (0.20 to 0.249 mm);

the thickness of the exterior yarn of the exterior part 622 in the second shape holding member 620 on the tip side as the thinner one is USP size 4-0 (0.15 to 0.199 mm); and

the thicknesses of the insertion yarn 614 and the insertion yarn 624 are USP size 6-0 (0.070 to 0.099 mm).

The exterior part 612 and the exterior part 622 are formed into the spiral (curl) shapes by winding the respective exterior yarns around metal bars having the diameter of 0.5 mm, for example, and thermally fixing them. The insertion yarn 614 and the insertion yarn 624 are inserted into the thus formed exterior part 612 and exterior part 622 having the spiral (curl) shapes, so that the first shape holding member 610 and the second shape holding member 620 are formed.

As illustrated in FIG. 14A and FIG. 14B, the first shape holding member 610 is provided on the root side of the defect hole closing material 100 and the second shape holding member 620 is provided on the tip side of the defect hole closing material 100. In this case, one end of the coil spring 140 is engaged with the first end part 112 (for example, caught on the loop of the filamentary material at the first end part 112), the other end of the coil spring 140 is engaged with the second end part 122 (for example, caught on the loop of the filamentary material at the second end part 122), and both ends of the coil spring 140 are respectively joined to the metal piece 412 and the metal piece 422 by caulking or the like, as described above. In addition, the first shape holding member 610 is coupled to the metal piece 412 on the root side, and the second shape holding member 620 is coupled to the metal piece 422 on the tip side. A coupling form between the metal pieces and the shape holding members will be described with reference to FIG. 14A and FIG. 14B and FIG. 15A to FIG. 15C.

FIG. 14A is a detailed view of the end part (on the tip side in this example) of the defect hole closing material 100 and FIG. 14B is a perspective view (virtual perspective view) thereof. FIG. 15A corresponds to FIG. 14A and is a more detailed view for explaining the coupling form between the metal pieces and the shape holding members, FIG. 15B is a view when FIG. 15A is seen from the back side of the page, and FIG. 15C corresponds to FIG. 15A and illustrates only the insertion yarns while the exterior parts are virtually detached. The lengths of the exterior parts are assumed to be the lengths for about the outer circumferences of the metal pieces for illustration in FIG. 14A and FIG. 14B and FIG. 15A to FIG. 15C for easy understanding of the coupling form between the metal pieces and the shape holding members in the defect hole closing material 100.

The lengths of the exterior parts and the positions of the exterior parts on the metal pieces are not particularly limited as long as the following action effects are provided and the shape of the defect hole closing material 100 can be held against the external force acting along the lengthwise direction of the defect hole closing material 100 for moving the defect hole closing material 100 (itself). For example, the lengths of the exterior parts may be the lengths for about the outer circumferences of the metal pieces, about half the outer circumferences of the metal pieces, or equal to or more than double of the outer circumferences of the metal pieces. In any of the cases, as illustrated in FIG. 13, regardless of the stretched states of the exterior parts, the exterior parts have the spiral shapes (curl shapes) and are easy to absorb the external force. Furthermore, they have the lengths smaller than those of the insertion yarns to cause the insertion yarns to be exposed from both ends of the exterior parts, and the shape holding members are coupled to the metal pieces using the exposed insertion yarns.

As for the coupling form between the metal pieces and the shape holding members, the coupling form between the first metal piece 412 and the first shape holding member 610 on the root side and the coupling form between the second metal piece 422 and the second shape holding member 620 on the tip side are the same. The following therefore describes the coupling form between the metal pieces and the shape holding members by using the first shape holding member 610 on the root side as a representative.

As illustrated in these drawings, although not limited, the first shape holding member 610 on the root side is coupled to the metal piece 412 by winding the exterior part 612 around the cylindrical metal piece 412 (a position illustrated as a position of the metal piece around which the exterior part is wound is merely an example), crossing the insertion yarn 614 on the tip side of the metal piece 412 for overhand knot, inserting a termination part 614E of the insertion yarn 614 into the cylinder inner side from the tip side of the metal piece 412, pulling out it through the through-hole 416 from the cylinder inner side to the outer surface side, and crossing the insertion yarn 614 for overhand knot.

Although not illustrated in FIG. 15A to FIG. 15C, the termination part 614E of the insertion yarn 614 travels toward the substantially central part 130 of the defect hole closing material 100, and the insertion yarn 614 is interlaced (intertwisted) with the filamentary material and the porous layer forming the defect hole closing material 100. That is to say, the insertion yarn 614 couples the filamentary material and the porous layer forming the defect hole closing material 100. Positional deviation of the porous layer in the defect hole closing material 100 can thereby be suppressed.

The case in which the defect hole closing material 100 is used for the catheterization for the atrial septal defect is described with reference to FIG. 6 to FIG. 9 for easy understanding of the above-mentioned action.

### [Usage Mode]

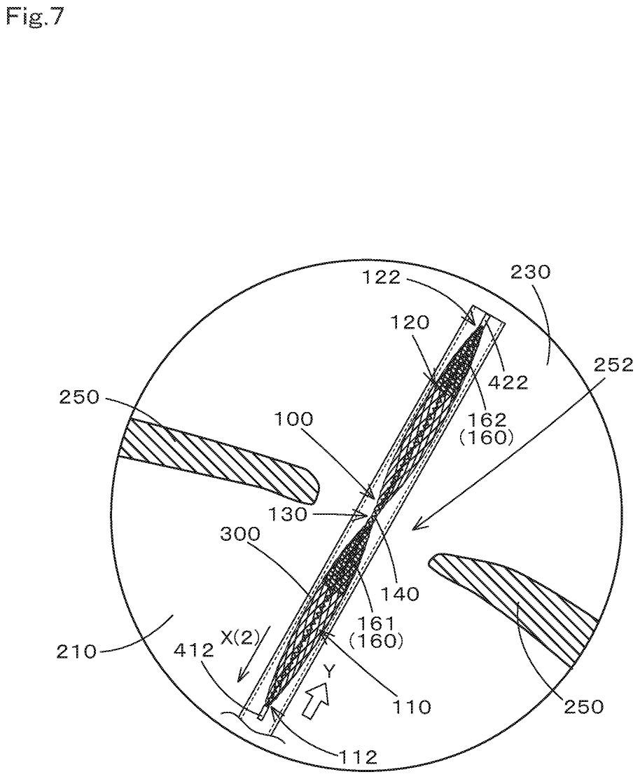

FIG. 6 is a conceptual view when the defect hole closing material 100 is used for the catheterization for the atrial septal defect, and FIG. 7 to FIG. 9 are enlarged views of a part B in FIG. 6 illustrating the procedure of the catheterization. In the following, only matters specific to the usage mode of the defect hole closing material 100 according to the embodiment are described. Since general matters are the same as those of well-known catheterization for the atrial septal defect, detailed description thereof is not repeated here.

As illustrated in FIG. 6, a heart 200 of a human includes two atria and two ventricles, i.e., the right atrium 210 connected to a superior vena cava and an inferior vena cava to receive venous blood from the whole body, a right ventricle 220 connected to the right atrium 210 via a pulmonary artery and a tricuspid valve 260 to feed venous blood to lungs, the left atrium 230 connected to a pulmonary vein to receive arterial blood from the lungs, and a left ventricle 240 connected to the left atrium 230 via an aorta and a mitral valve 270 to feed arterial blood to the whole body. The atrial septal defect is a disorder in which the defect hole 252 opens in an atrial septum 250 separating between the right atrium 210 and the left atrium 230. Note that in FIG. 6, the tip side of the catheter 300 is indicated by a virtual line and the defect hole closing material 100 contained in the catheter 300 is indicated by a solid line for easy understanding.

First, outside the living body, the first end part 112 and the second end part 122 of the defect hole closing material 100 that expands to a size appropriate for the defect hole 252 are pulled in mutually separating directions, so that the whole of the coil spring 140 is stretched to make the cylinder diameter of the defect hole closing material 100 including the porous layers 160 (the first porous layer 161 and the second porous layer 162) smaller than the inner diameter of the catheter 300. Thus, the defect hole closing material 100 is set in the catheter 300. The catheter 300 containing the defect hole closing material 100 is inserted from the femoral vein (see FIG. 3) and is moved in the direction indicated by an arrow an X(1) to pass through the defect hole 252 from the right atrium 210 side, so that the catheter 300 containing the defect hole closing material 100 is brought close to the left atrium 230 side.

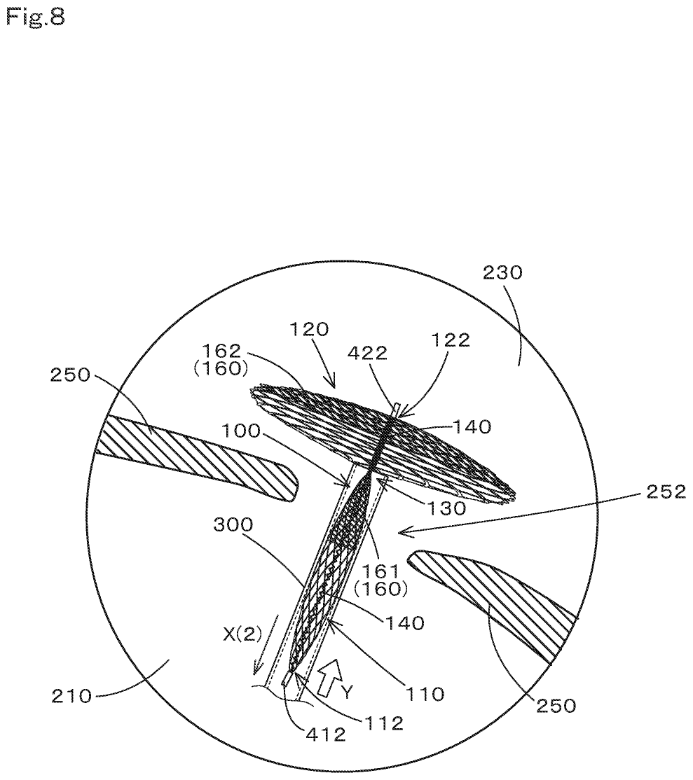

As illustrated in FIG. 6 and FIG. 7, at a position where the substantially central part 130 of the defect hole closing material 100 faces the vicinity of the defect hole 252, the catheter 300 containing the defect hole closing material 100 is stopped. In the living body, when pushing the second cylinder part 120 out of the catheter 300 with the applicator or the like in the direction indicated by the arrow Y, the second cylinder part 120 the shape of which has been restricted by the inner wall 310 of the catheter 300 can freely change its shape, and only the part of the coil spring 140 that is contained in the second cylinder part 120 is compressed to only increase the cylinder diameter of the body part of the second cylinder part 120 and expand the second porous layer 162 as illustrated in FIG. 8.

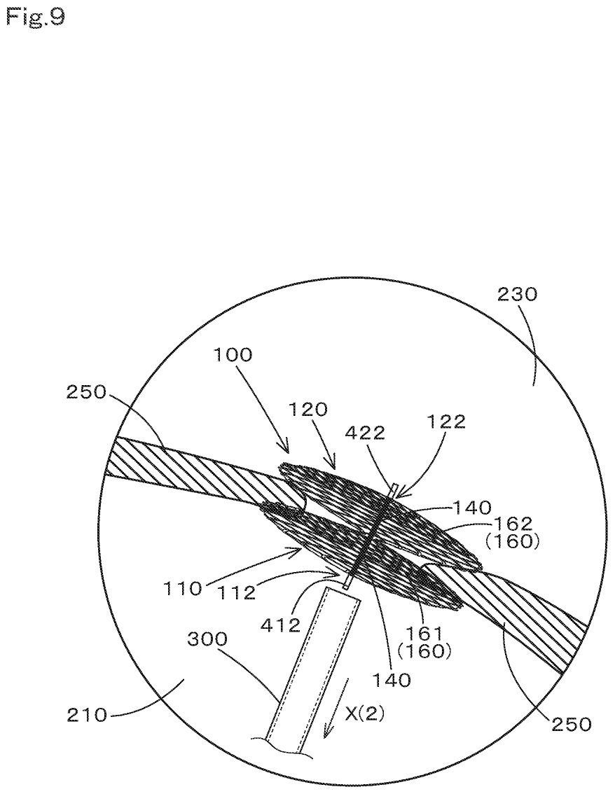

When further pushing the first cylinder part 110 out of the catheter 300 with the applicator or the like in the direction indicated by the arrow Y, the first cylinder part 110 the shape of which has been restricted by the inner wall 310 of the catheter 300 can also freely change its shape, and the part of the coil spring 140 that is contained in the first cylinder part 110 is also compressed to increase the cylinder diameter of the body part of the first cylinder part 110 and expand the first porous layer 161 as well, as illustrated in FIG. 9.

That is, when pushing the defect hole closing material 100 out of the catheter 300 with the applicator or the like, the second cylinder part 120 and the second porous layer 162 arranged on the left atrium side first expand, and then, the first cylinder part 110 and the first porous layer 161 arranged on the right atrium side expand. As a result, the first cylinder part 110 and the first porous layer 161 arranged on the right atrium 210 side and the second cylinder part 120 and the second porous layer 162 arranged on the left atrium 230 side come close to each other with the substantially central part 130 (the defect hole 252) as the center, and also the first cylinder part 110 and the first porous layer 161 and the second cylinder part 120 and the second porous layer 162 expand. Finally, as illustrated in FIG. 9, the first cylinder part 110 and the first porous layer 161 and the second cylinder part 120 and the second porous layer 162 sandwich the atrial septum 250 from both side thereof, and the defect hole 252 opening in the atrial septum 250 can be occluded by the defect hole closing material 100.

After that, the catheter 300 is moved in the direction indicated by an arrow X(2) to take the catheter 300 out of the living body, thereby completing the treatment. With this treatment, in the living body (to be accurate, near the defect hole 252), the defect hole closing material 100 that is wholly made of the bioabsorbable material (the coil spring 140, the metal piece 412, and the metal piece 422 are excluded in some cases) is placed. As described above, since all of the materials of the defect hole closing material 100 placed in the living body are the bioabsorbable material (the coil spring 140, the metal piece 412, and the metal piece 422 are excluded in some cases) and finally absorbed in the living body, there is almost no fear of long-term failure.

In addition, when the coil spring 140 is not provided, it is necessary to fix the form of the defect hole closing material 100 to the form illustrated in FIG. 9 before the defect hole closing material 100 is placed in the living body. As a conceivable method, for example, heat fusibility is imparted to the bioabsorbable fiber 150, and the bioabsorbable fiber 150 is thermally set within the living body. The defect hole closing material 100 is however advantageous because the coil spring 140 enables the form of the defect hole closing material 100 to be fixed to the form illustrated in FIG. 9.

In such a usage mode, when pushing the first cylinder part 110 and the second cylinder part 120 in this order out of the catheter 300 with the operation wire 500 in the direction indicated by the arrow Y, external force (push-out force with the operation wire 500) is made to act along the lengthwise direction for moving the defect hole closing material 100 along the lengthwise direction. When the external force is made to act, a defect hole closing material including no shape holding member cannot hold the shape of the defect hole closing material in some cases as illustrated in FIG. 16B.

By contrast, when the defect hole closing material 100 including at least one shape holding member is used, the following advantage can be provided. That is, even when the external force can be made to act only from the root side of the defect hole closing material 100, the shape holding member including the exterior part having the spiral shape (curl shape) and the insertion yarn coupling the exterior part to the metal piece can prevent deformation of the shape of the defect hole closing material 100 due to deformation of the first cylinder part 110 even with action of the external force, thereby holding the shape of the defect hole closing material 100.

As described above, since the defect hole closing material 100 according to the embodiment is wholly made of the bioabsorbable material (the coil spring 140, the metal piece 412, and the metal piece 422 are excluded in some cases) and is finally absorbed in the living body, there is almost no fear of long-term failure. Inclusion of the coil spring 140 enables the cylinder diameter of the defect hole closing material 100 to be easily changed together with the porous layers. The defect hole closing material 100 can thereby be easily set in the catheter by changing the cylinder diameter of the defect hole closing material 100 and the sizes of the porous layers to be decreased. Furthermore, when the coil spring 140 is included, only by pushing the defect hole closing material 100 out of the catheter 300 at a position of the defect hole, the cylinder diameter of the defect hole closing material 100 can be easily changed to be increased together with the porous layers such that the two cylindrical bodies come close to each other, and the form can therefore be easily fixed, thereby occluding the defect hole opening in the atrial septum.

The defect hole closing material 100 according to the embodiment includes the shape holding members. Accordingly, even when the external force along the lengthwise direction of the defect hole closing material 100 acts for moving the defect hole closing material 100, the shape of the defect hole closing material 100 can be held against the external force, thereby occluding the defect hole opening in the atrial septum accurately with preferable operability.

Note that the embodiment disclosed herein should be considered to be illustrative in all respects and non-limiting. The scope of the present invention is indicated by the appended claims rather than the foregoing description, and all changes that come within the meaning and the range equivalent to the claims are intended to be encompassed therein.

### INDUSTRIAL APPLICABILITY

The present invention is suitable for use as a medical material which is set in a catheter for treatment of a defect hole formed in a biological tissue and is particularly preferable in that the medical material is capable of being released and placed at a treatment site, enables low invasive treatment, has almost no fear of long-term failure even when the medical material remains in the body, and is preferable in operability.

### REFERENCE SIGNS LIST

100 MEDICAL MATERIAL (OCCLUDER)
110 FIRST CYLINDER PART
112 FIRST END PART
120 SECOND CYLINDER PART
122 SECOND END PART
130 SUBSTANTIALLY CENTRAL PART
140 COIL SPRING
150 BIOABSORBABLE FIBER
160 POROUS LAYERS (FIRST POROUS LAYER 161, SECOND POROUS LAYER 162)
200 HEART
250 ATRIAL SEPTUM
252 DEFECT HOLE
300 CATHETER
610, 620 SHAPE HOLDING MEMBER

1. A medical material that is formed by a cylindrical body having a stitch-like structure using a filamentary material, wherein
the medical material has such a shape that a cylinder diameter of a substantially central part of the cylindrical body is smaller than cylinder diameters of the other parts,
a first cylinder part on a first end part side as one end of the cylindrical body in a lengthwise direction and a second cylinder part on a second end part side as the other end of the cylindrical body are formed with the substantially central part as a center,
an elastic member both ends of which are respectively engaged with the filamentary material at the first end part and the filamentary material at the second end part and that is made to pass through inner parts of the first cylinder part and the second cylinder part from the first end part side to the second end part side via the substantially central part is included,
at least any one of a first porous layer arranged on the substantially central part side in the first cylinder part and a second porous layer arranged on an opposite side to the substantially central part in the second cylinder part is arranged on an inner surface of the cylindrical body, the first porous layer and the second porous layer being formed by any one of a nonwoven fabric, sponge, film, and composite body of the nonwoven fabric, sponge, and film, and
a shape holding member that is provided at at least one of both ends of the elastic member, is coupled to an end part of the elastic member, and holds the shape of the medical material against force acting along the lengthwise direction for moving the medical material is included.

2. The medical material according to claim 1, wherein the shape holding member includes an exterior part formed by winding a yarn into a spiral shape.

3. The medical material according to claim 1, wherein the shape holding member includes an exterior part formed by winding a yarn into a spiral shape and an insertion yarn inserted so as to pass through a center of the spiral shape.

4. The medical material according to claim 3, wherein when the shape holding members are provided at both ends of the elastic member, thicknesses of the yarns forming the exterior parts are made different.

5. The medical material according to claim 4, wherein when the medical material is contained in a catheter, the yarn forming the exterior part in the shape holding member on a root side is made thicker than the yarn forming the exterior part in the shape holding member on a tip side.

6. The medical material according to claim 3, wherein in at least any one of the cylinder parts, the insertion yarn couples the filamentary material and the porous layer.

7. The medical material according to claim 4, wherein in at least any one of the cylinder parts, the insertion yarn couples the filamentary material and the porous layer.

8. The medical material according to claim 5, wherein in at least any one of the cylinder parts, the insertion yarn couples the filamentary material and the porous layer.
